(19) United States
(12) Patent Application Publication
(10) Pub. No.: US 2013/0204226 A1
(43) Pub. Date: Aug. 8, 2013

(54) Pressure Actuated Catheter Valve

(71) Applicant: I-V Access Technology, Inc., Fresno, CA (US)

(72) Inventor: Stephen Richard Keyser, Fresno, CA (US)

(73) Assignee: I-V Access Technology, Inc., Fresno, CA (US)

(21) Appl. No.: 13/759,643

(22) Filed: Feb. 5, 2013

Related U.S. Application Data
(60) Provisional application No. 61/595,415, filed on Feb. 6, 2012.

(51) Int. Cl. A61M 39/26; A61M 25/00

(52) U.S. Cl. CPC A61M 39/26; A61M 25/0097
USPC 604/506; 604/167.04; 604/164.1; 137/511; 137/2

(57) ABSTRACT

This invention provides catheter assemblies and methods for insertion of a catheter into a vessel. The assemblies include a resilient valve in the hub of the catheter that can be opened by pressure from a male luer fitting. The methods include piercing a vessel with the needle and catheter of the assembly, retraction of the needle component thus closing the resilient valve. Finally the catheter can be accessed by applying a male luer fitting to the catheter hub and a shoulder of the resilient valve.

### CROSS-REFERENCE TO RELATED APPLICATIONS

This application claims priority to and benefit of a prior U.S. Provisional Application No. 61/595,415, Pressure Actuated Catheter Valve, by Stephen Keyser, filed Feb. 6, 2012. The full disclosure of the prior application is incorporated herein by reference.

### FIELD OF THE INVENTION

The invention is directed to methods and devices to facilitate sanitary connection of luer devices to inserted catheters. Catheter assemblies include resilient valves having a distal tapered side with slits defining one-way flaps toward the catheter. The proximal side or the valve can have a first recess with an inner shoulder defined by a second recess centered in the first recess. When pressure is applied to the shoulder, the valve flaps in the second recess can pivot out at pivot points in the first recess, providing an unobstructed fluid flow path past the flaps. In the methods, the valve can hold and seal in place a needle and dilator in the catheter, while the catheter is being placed in a blood vessel. The needle and dilator can then be removed from the catheter, sliding past the valve. The valve can then close to provide a sanitary seal for the catheter, while offering an easy open fluid flow path for, e.g., a male luer fitting pressing on the valve proximal side shoulder.

### BACKGROUND OF THE INVENTION

Catheters are inserted into medical patients to provide continuous access to the patient's blood stream. However, this point of access must remain sanitary, while allowing ready coupling with any number of devices, e.g., operating to inject a fluid into the patient or to draw a blood sample from the patient.

In a typical configuration, a catheter needle will have a proximal female luer fitting which is connected to a male luer fitting of an intravenous (IV) fluid line. A Y intersection with a piercible septum can be provided in the IV line for fluid access with a needle and syringe. Drugs can be administered to the patient by injecting the drug into the IV fluid stream using a syringe and needle at the Y-branch septum. Of course, such an arrangement introduces a poorly flushed dead leg in the system that interferes with fluid transfers and can present an environment for microbial growth. The Y intersection can also be used to draw a sample of the patient's blood from the catheter, again using a syringe and needle through the septum. However, these operations are problematic. As a preliminary matter, the operations require manipulation of a needle, with its associated hazards. In addition, in many cases it can be undesirable to have the drug diluted in the IV fluid during injection, or a blood sample diluted with IV fluid as it is withdrawn.

To avoid the use of needles in injection and sampling operations through catheters, devices exist wherein access is available directly using a male luer fitting connection to the catheter. In Luer Activated Device with Minimal Fluid Displacement, U.S. Pat. No. 7,753,338, to Desecki, luer sealing valves include an inlet seal adapted to receive a male luer, and an outlet valve adapted so that a male luer inserted into the aperture will open fluid flow through the outlet. However, the devices cause significant fluid displacement when the luer is pushed through the first seal, and can leave possibly contaminated fluids in the space between seals as the luer fitting is withdrawn. Further, the multiple seals are unduly complex, and increase the forces required to make the necessary connections. In addition, the Desecki valve designs include a bulk of hardware in the flow path of the valve that can occlude fluid flow and increase forces necessary to open the valve.

U.S. Pat. No. 7,736,339 Woehr et.al. uses a flat disc of silicone as a valve and use a ‘valve actuating element’ (a hollow conically shaped plastic part that is pushed by a male luer fitting) into the valve disc opening a central orifice as the actuating element is forced through. Such an arrangement can allow blood to be trapped between the distal side of the Woehr valve and the catheter hub.

In light of these problems, it would be beneficial to have relatively simple luer connection for catheters (and other types of conduits or vessels) that minimize fluid displacement and dead volumes. It would be desirable to have a quick, resealable, low force connection to a vessel, while preserving a sanitary seal once the connection is severed. The present invention provides these and other features that will be apparent upon review of the following.

### SUMMARY OF THE INVENTION

The present invention relates generally to the field of peripheral vascular access and to an apparatus whereby simple luer connectors can readily gain access to an IV catheter to draw or inject fluids.

Catheter assemblies of the invention can include a resilient self-closing valve adapted to open with pressure from the end of a conduit (e.g., male luer fitting) at the proximal side of the valve. For example, the self closing valve can include a tapered distal surface, a proximal surface with a first recess, a second recess in the first recess above (distally or efferently) the first recess, and one or more slits running from the distal surface to the second recess. The first and second recesses can have circular outer edges, e.g., with the outer edge of the second recess defining a stepped shoulder between the first and second recesses.

In many embodiments, the one or more slits run from a top center of the distal surface to the proximal surface at least to the edge of the second recess where the second recess contacts the first recess. Typically, the one or more slits run to a point between the shoulder and the outer edge of the first recess. In certain embodiments, the valve distal tapered surface is conical and the slits comprise three or more radially arranged slits. The valve bottom (proximal) surface can include a flange around the first recess. In preferred embodiments, the self closing valve is made from a resilient material. In preferred embodiments, the valve is configured so that a force exerted at an intersection (shoulder region) of the first and second recesses forces opens the one or more slits (and flaps they define), thereby providing a fluid flow path through the second recess.

A full catheter assembly can include a catheter and a resilient valve of the invention. The assembly can further include other features, e.g., that aid in the insertion of the catheter into a vessel. For example, a catheter insertion assembly can include a catheter comprising a hub at a catheter proximal end, and a resilient self-closing valve in the hub having a distal tapered surface and a proximal surface comprising a first recess, wherein the first recess comprises a second recess located on the surface of the first recess. The assembly can include a flexible dilator and/or rigid needle slidably mounted within the catheter through the valve. The resilient valve can be configured to close and seal to fluid flow when the dilator and/or needle are withdrawn from the valve.

The valve in the catheter assembly can be as above. For example, the second recess can be located on the distal center of the first recess. The one or more slits can run from a top center of the valve distal surface to, e.g., at least an outer edge of the second recess at a point where the second recess contacts the first recess. The one or more slits can include, e.g., at least 3 slits in a radial pattern. In many embodiments, the catheter hub includes a female luer fitting just proximal to the resilient valve.

The present inventions include, e.g., methods of accessing fluids from a catheter emplaced in a vessel. For example, a method of accessing a catheter or needle bore can include providing a catheter or needle hub comprising a self-closing resilient valve. The valve can comprise: a tapered distal surface, a proximal surface with a first recess and a second recess (wherein the second recess is located distally in the first recess defining a shoulder at an intersection of the first and second recesses), and one or more slits running from the distal surface to the second recess. In use, access to the catheter bore can be obtained by pushing an end of a conduit distally onto the valve shoulder to open the valve, thus providing a fluid flow path between the bore at the distal surface and the conduit at the proximal surface. Typically, the conduit is a male luer fitting.

In preferred embodiments of the methods, the valve second recess can have a circular outer edge centered in the first recess. Further, the first recess can have an outer edge on the proximal surface, and the one or more slits can be configured to run to a point between the shoulder and the outer edge of the first recess.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “fluids” can include mixtures of fluids, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “vessel”, as used herein, refers to a chamber in which a fluid is held or a conduit through which a fluid travels. For example, a typical vessel can be a blood vein, artery, or lymph vessel. In some aspects of the invention, a vessel can be a segment of the digestive tract, a gland duct or a cerebral-spinal fluid chamber. In a more generic context, a vessel can be a chamber (such as a container or storage vessel) or a conduit (such as an industrial pipe or hose) for transferring a fluid.

The “distal” end of a device component is the end closest to the patient or target vessel, in use, e.g., the end of the component directed toward the vessel or intended to enter a vessel first. For example, the distal end of a guide needle is the piercing end. The distal end of a dilator or catheter is the end intended to be inserted into a patient's skin or vessel. The distal surface of a resilient valve is, e.g., the outwardly tapered surface directed toward the distal tip of the catheter. Note, however, that the components of the present valves can be reversed and still function, e.g., with fluids flowing in either direction through the opened valve.

The “proximal” end is the end of the device component oriented opposite the distal end. For example, the proximal end of a catheter insertion assembly can include the ends of the components not intended for insertion into the patent, such as the dilator hub end, or guide needle hub end. The proximal surface of a resilient valve is, e.g., the surface on the side of the valve directed outward, away from the vessel, in use. Typically, the proximal end is the end directed toward the technician user, and the distal end is the end directed to the vessel.

A “recess”, as used herein, is a concavity or depression on a surface. A second recess on a first recess is a concavity on the surface of the first recess. The second recess is typically entirely within the boundaries of the first recess, i.e., the outer edge of the second recess does not extend beyond the outer edge of the first recess. The “edge” of a recess is as one would expect, e.g., where the recess transitions into the surface into which it is depressed.

A needle is said to be “retracted”, e.g., within a dilator, when the needle is repositioned proximally relative to the dilator. Retracted needles are typically retracted proximally at least to the point where the piercing tip is within the dilator.

A “resilient” material tends to return to its original position when a deforming force is removed. A resilient seal typically comprises a seal formed when a device component is forced to slide through and deform a resilient seal component, so that the resilient component is urged against the surface of the device component leaving no space in between. Typical resilient seals include resilient valves, septa, sleeves and/or o-rings. A resilient dilator or catheter is characterized by an ability to resiliently flex and bend along the central axis, e.g., to reduce physical stresses at an insertion site and/or to conform with the path of a vessel into which they are inserted. Resilient valves have resilient valve flaps that return to a normally closed position, e.g., when pressure of a conduit is removed from the proximal side of the valve.

Components of a catheterization device are “slidably mounted”, e.g., when inner and outer components can be axially rotated and/or axially translated relative to each other.

An “axis”, as typically used herein, is an imaginary line parallel to and in the center of a tubular device, for example, the center of radial symmetry. The term axial or axially thus refers to the direction that runs parallel to the axis, e.g., of a tubular device.

Two components are concentric when their major axes are coincident.

A beveled tip is the tip of a guide needle that is formed by a diagonal cut across the distal end of the needle, forming a sharpened edge that is used for piercing.

A typical guide dilator is a long, slender, tubular device, usually made of a flexible plastic, that fits concentrically over a guide needle so that the inside diameter of the guide dilator contacts the outside diameter of the guide needle and typically can slide over the guide needle. A guide dilator is typically mounted within a catheter intended for placement in a vessel, e.g., with the dilator removed after the placement.

A hub is a part of a catheter, dilator or guide needle at the proximal end, which typically flares out to a larger internal diameter, or at least a larger external diameter. The hub can provide a base for manipulating, mounting or employing features, such as detents or needle retractor devices, catches, valves, etc. The hubs can provide functional interaction (e.g., connection) of the catheter or catheterization device with external devices, such as, e.g., trocars, syringes, fluid administration lines, optic fibers, vacutubes, etc. Hubs can provide mounting positions for resilient valves.

An intra-vascular catheter is typically as is understood in the art. The IV catheter can include a long, slender, tubular body, usually made of a flexible plastic that fits concentrically over a guide dilator or needle so that the inside diameter of the intra-vascular catheter contacts the outside diameter of the guide dilator. Optionally, the catheter assembly does not include a needle or dilator, but has a piercing end for insertion without a separate needle.

A guide needle is a tubular device, e.g., usually made of stainless steel, that has a sharpened tip at its distal end that is used to puncture the skin and a targeted blood vessel, creating a hole through which a catheter may be guided. In the catheterization devices of the invention, the needle is typically a guide needle concentrically and slidably mounted, e.g., within a guide dilator, which in turn, is mounted within the catheter.

A tapered tip, in the context of the invention, is a tip of a catheter, dilator or guide needle whereby the outside diameter of the tube decreases approaching the distal end, thus making the tube wall thinner. During piercing or insertion through skin or a vessel wall, a tapered tip can facilitate expansion of a pierced hole from one diameter to a larger diameter.

A valve, e.g., controls fluid flows to, from or within a conduit. A self-closing valve returns to a closed position once relieved of external forces. A one-way valve allows fluid flow in one direction but seals against flows in the opposite direction. Valves of the inventions include, e.g., special features, as described herein.

A flash cup is a mechanical feature that may be incorporated into the guide needle hub, allowing the caregiver to detect when the vessel wall has been punctured by virtue of vessel fluid filling the flash cup chamber.

### BRIEF DESCRIPTION OF THE DRAWINGS

The drawings here shown include exemplary embodiments of the invention. It is to be understood, however, that the present invention may be embodied in various forms. Some aspects of the invention may be shown exaggerated or enlarged in the drawings to facilitate an understanding of the invention.

FIG. 1A is schematic diagram of a resilient valve in the normally closed position. FIG. 1B shows an alternative embodiment wherein the valve apex is foreshortened, e.g., without a conical point apex, and including a split torus on the top and/or bottom surfaces of the flange.

FIG. 2 is a cross sectional schematic diagram of a resilient valve forced open by contact of the valve shoulder with the end of an external conduit.

FIG. 3 is a schematic diagram of a catheter assembly including a needle, dilator and a catheter with a self-closing valve in the hub.

FIG. 4 is a typical valve of the invention.

### DETAILED DESCRIPTION

The present inventions are directed to devices and methods to facilitate access to fluids from an IV catheter. The catheters include resilient valves that allow nesting and sealing of, e.g., needle and dilator features in the catheter. The valves provide sanitary sealing when such features are withdrawn, and provide easy access to the catheter bore, e.g., when contacted by a male luer fitting. The methods of accessing catheter fluids can employ devices of the invention and sequential steps to access a catheter bore, e.g., after it is placed in a blood vessel of a patient.

The catheter insertion devices typically include a three-part compliment of concentric conduits to pierce and dilate an insertion point into a vessel for ultimate insertion and placement of a catheter. The catheterization device can have a needle and dilator slidably inserted into a catheter through a resilient valve. The valve can be structurally adapted to hermetically seal the catheter bore when the needle and dilator are withdrawn from the catheter. The valve can be configured to open, providing a fluid flow path, when pressure is applied to the proximal surface, as will be discussed in detail below. Although the present inventions are generally discussed in the context of, e.g., a catheter/dilator/needle assembly, the inventions are not so limited, and can be employed more generally.

Methods of the invention generally include insertion of a catheter assembly into a vessel, removal of the dilator and needle, resilient sealing of the valve, and access to the catheter bore by contacting the valve with the distal end of a male luer fitting. When the luer fitting is withdrawn, the valve again seals against fluids from the catheter bore.

Methods of placing catheters can include provision of a catheter insertion device, piercing a clinical patient's skin and/or vessel, dilating the pierced point and positioning the catheter within the vessel. The catheter insertion device can include three complimentary concentric conduits with features and functions that facilitate catheter placement. For example, a rigid sharp needle can be slidably mounted within a flexible dilator layer having a tapered tip and mounted within a catheter through a self sealing valve. The needle can pierce and initially dilate a hole in the wall of a vessel. The device can be urged forward so that the tapered end of the dilator can smoothly intrude into the hole and expand the hole circumference. Once the dilator is within the vessel, the needle can optionally be retracted or withdrawn entirely from the device. The dilator can support entry of a tapered catheter distal end into the vessel through the hole. The dilator can optionally be inserted some distance along the internal lumen of the vessel with minimal risk of trauma to the vessel interior and provide a guide for extended insertion and placement of the catheter. Ultimately, the dilator can be withdrawn from the catheter and valve. With the needle and dilator withdrawn from the valve, it springs to a normally closed condition, preventing fluids from leaking from the catheter bore, while providing easy access to the catheter using any external device having the appropriate, e.g., male luer connector.

### Catheter Insertion Devices With Resilient Access Valves

As mentioned above, the catheter assemblies generally include a catheter having a resilient valve in a proximal hub. A piercing needle (and optional dilator) is positioned through the valve, slidably mounted through the bore of the catheter. The piercing guide needle is typically rigid and hollow with a sharp distal tip for initial penetration of a vessel wall. The distal ends of the needle and catheter are usually tapered to smoothly expand a point of entry into the vessel as the device is pushed into the vessel. The proximal end includes a radially expanded hub holding a resilient valve. The hub can be used to manipulate the catheter components, to provide mounting structures for device accessories, and/or to provide connections to external conduits and devices. When the catheter is emplaced and needle withdrawn, the resilient valve can control flow of fluids into and out from the catheter bore.

Self Closing Valves

Resilient valves of the invention typically include a tapered distal surface with slits running to a proximal valve surface. Generally, the proximal surface has paired concentric recesses with a stepped shoulder between. The shoulder can receive the flat end of a conduit (e.g., male luer fitting) and act as a lever to force open the distal surface at the slits. The shoulder can also seal the end of the conduit.

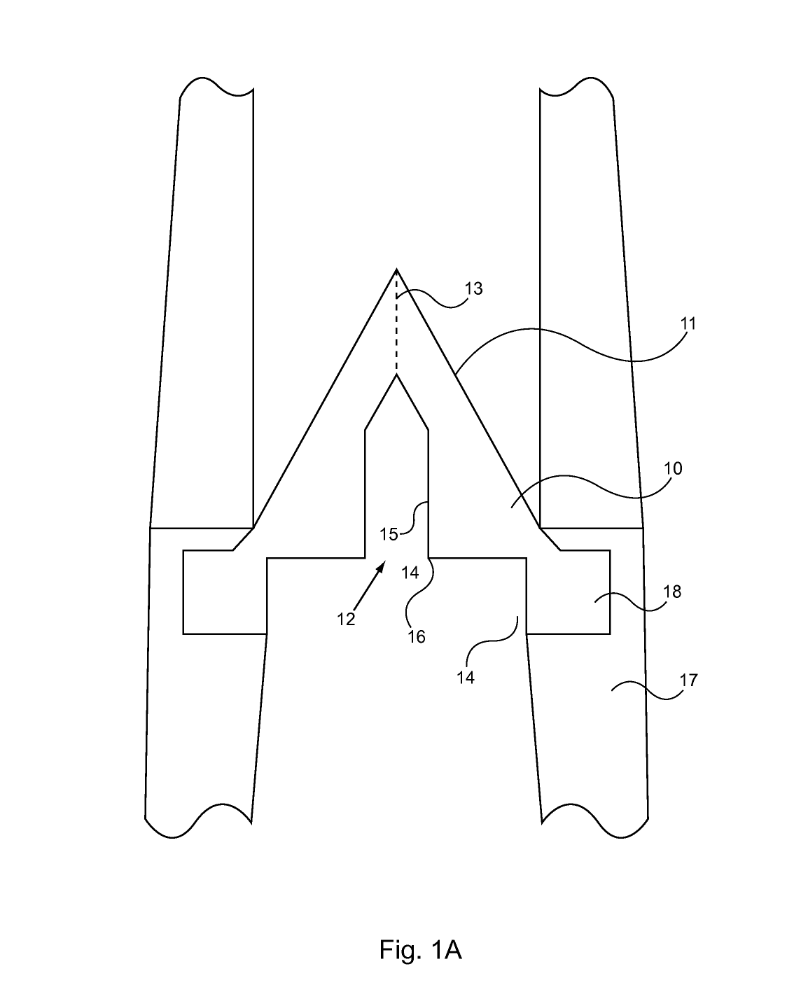

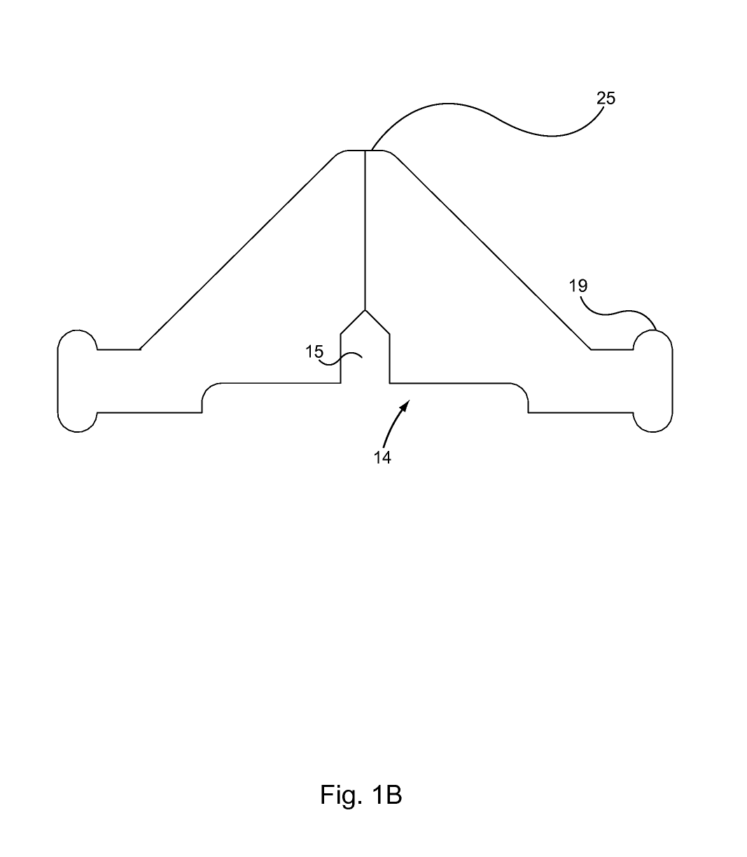

For example, as shown in FIG. 1A, valve 10 has distal surface 11 and proximal surface 12. One or more slits 13 run through the valve between the distal and proximal surfaces. The proximal surface has a first recess 14 and second recess 15 defining shoulder 16. In many embodiments, the valve is mounted within catheter hub 17. Alternately, the valve can be without the first recess, e.g., with the second recess running from approximately the level of the flange bottom, instead of starting from a first recess.

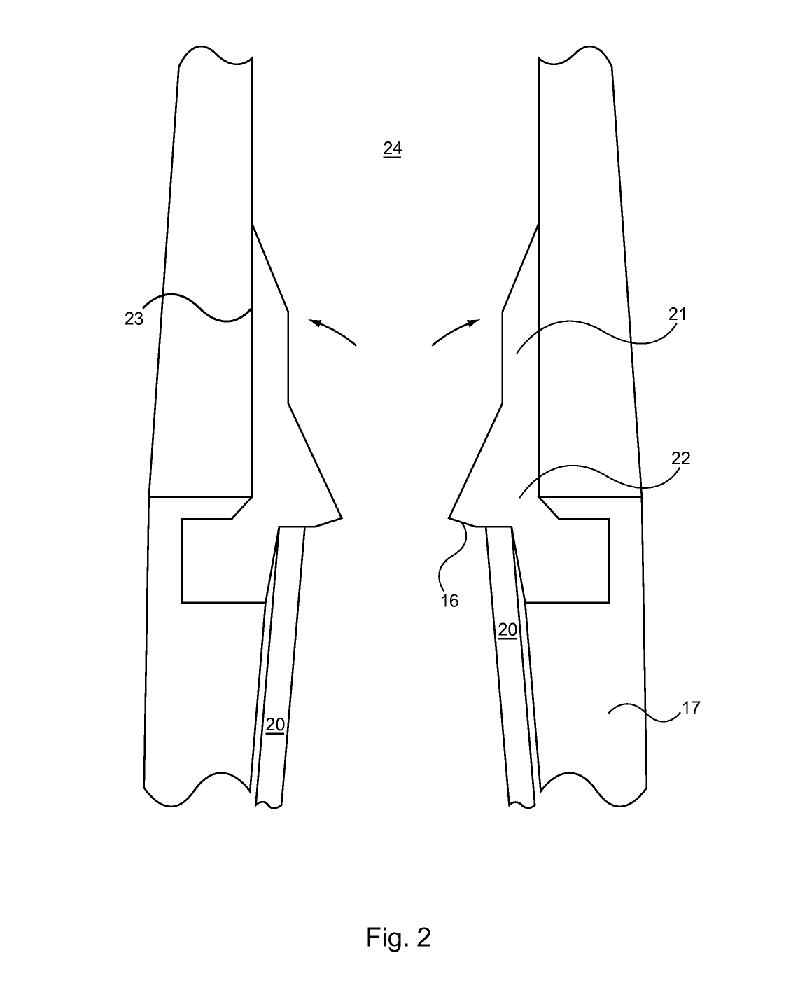

In use, as shown in FIG. 2, a male luer connector 20 is inserted into female luer fitting of hub 17 to a point where the distal face of the male luer contacts shoulder 16 causing the flaps 21 to swing outwardly on pivot region 22, contacting inner wall of catheter 23 providing an unobstructed flow path between the male luer and catheter bore 24. Note that the second recess not only defines the shoulder in the first recess, but also allows flap pivoting with reduced force and substantially reduces obstruction, providing for fluid flows with less resistance and less turbulence than old art designs.

In another embodiment, the geometry at the apex of the distal side of the valve can have, e.g., a flat or curvilinear apex 25 (as shown in FIG. 1B). Such a design can make it easier to produce the slits in manufacturing. Such a trimmed topography can make it easier to obtain a perfect cut at the apex during the manufacturing process, especially with a soft elastic material. The resulting slit can be more complete and well-centered, e.g., allowing the valve to close properly and avoiding the possibility that a fragment or burr of uncut material to can create turbulence of escape into a blood vessel.

The valves of the invention are not limited to use in medical catheters. Therefore, the general concepts can be applied to any number of different situations. The vessels accessed by the “catheter” are not limited to only vessels and chambers of living organisms. For example, the valves can be used in industrial processing environments where it is desirable to provide ready access to a first conduit by a second conduit, or access to a chamber by a pipe or hose (wherein the chamber is adapted with a valve of the invention). In certain embodiments, the vessels accessed through the valves can be, e.g., a catheter, a dilator, a chamber, a pipe, a hose, a storage tank, a carboy, a fuel tank, a hydraulic line, and/or the like.

The valves include a tapered distal surface and a proximal surface with a second recess within a first recess. The tapered distal surface is preferably uniformly tapered, and often generally conical, e.g., to provide a complimentary contact and minimum dead volumes between the valve flaps and the catheter bore, when the valve is in the opened position. The cross sections (across the axis of fluid flow or the axis of the catheter bore) of the recesses can be any shape, as functionally appropriate. However, in preferred embodiments, one or both of the recesses have a round cross section. The aspect ratio (axial length over width of base) of the valve tapered surface is preferably about 1:1. However, the aspect ration can range from less than 1:10 to more than 10:1, from 1:5 to 5:1, from 2:1 to 1:2, from 1.5:1 to 1:1.5, or about 0.8:1. Preferably, the cross sections are concentric. This structural configuration can provide many advantages, such as, e.g., complimentary contact with typical conduit (e.g., male luer) ends, uniform flap opening, and less turbulent fluid flows.

The valves are typically flap valves with a resilient bias to a normally closed position. Optionally, the valves are closed by a hydraulic back pressure from the distal side of the valve. The valve flaps are defined by the boundaries of the slits and the pivot region. The pivot region, e.g., between the outer extent of the shoulder lever action arm and the nearest surface of the valve distal surface, can range in thickness from, e.g., more than 5 cm to less than 0.1 mm, from 1 cm to 0.2 mm, 5 mm to 0.25 mm, 1 mm to 0.3 mm, or preferably about 0.4 mm. Optionally, the pivot region can be hinged. The pivot region can become somewhat compressed when the conduit is applying force to the valve shoulder. The pivot region can range in thickness from more than 50% the shoulder lever action arm to less than 1%, from 40% to 10%, from 25% to 15%, or preferably about 20% of the shoulder action arm length.

The valve can include a flange 18 useful in mounting the valve in the catheter hub, e.g., between the catheter body and the hub body. The flange can provide a hermetic seal and maintain the valve in a functional relationship relative to the other assembly components, and relative to an external conduit requiring access to the catheter bore. In some embodiments, the inner surface of the flange can effectively be part of the first recess surface. In certain embodiments, it can be beneficial to include a ring 19 (e.g., a split torus) of material near the outer diameter of the top and/or bottom surfaces of the flange. Such an additional ring can provide additional resistance to prevent the valve from being drawn out of its position in the catheter hub groove, e.g., from the shear forces applied by the luer fitting when the valve is opened. To further secure the fit of the valve in the hub, the hub groove can include complimentary ring seats in the upper and/or lower groove surface to receive the additional ring(s).

In many embodiments, the valve can range in width, across the proximal surface, from more than 20 cm to less than 0.5 mm, from 10 cm to 1 mm, from 1 cm to 1.5 mm, from 8 mm to 2 mm, or about 5.5 mm. The diameter of the first recess can then range, e.g., from more than 20 cm to less than 0.5 mm, from 10 cm to 1 mm, from 1 cm to 1.5 mm, from 5 mm to 2 mm, or preferably about 4 mm. The diameter of the second recess can then range, e.g., from more than 20 cm to less than 0.3 mm, from 10 cm to 0.5 mm, from 1 cm to 0.6 mm, from 5 mm to 0.7 mm, 3 mm to about 0.8 mm, or preferably about 0.9 mm. Optionally, there is no first recess and the “second recess” originates at a surface described by the proximal plane of the flange. The cross section of the second recess is less than the cross section of the first recess, thus allowing for the shoulder area. The diameter of the second recess typically ranges from about 90% of the first recess diameter to about 5% of the first recess diameter, from about 80% to 10%, from 50% to 15%, or about 20% or 25% of the diameter of the first recess. The diameter of the first recess typically ranges from about 98% of the overall proximal surface diameter to about 10% of the overall proximal surface diameter, from about 95% to 50%, from 90% to 70%, or about 80% of the diameter of the overall proximal surface recess. If the valve shape is not conducive to diameter measurements, the above can be considered as relative surface area comparisons or cross-sectional area comparisons. In some preferred embodiments, the diameter of the second recess is about the same as the dilator or needle inserted through the valve in any particular catheter assembly, e.g., to provide a low friction fluid seal.

The valves can be made from any appropriate material. Typically, the valves are made from a resilient material, such as, e.g., a rubber, silicone rubber, a flexible plastic, or other resilient polymer. It is envisioned that valves with the above functions can be made from spring loaded mechanically hinged solid materials, but this is typically less preferred for reasons of manufacturing simplicity, reliability and sanitation. From a materials standpoint, an elastomeric material with higher modulus and higher hardness provides higher valve closure forces but reduces seal characteristics. A lower modulus and lower hardness material conversely provides a better seal surface but lower closure force. So for a single homogeneous material. In some embodiments, a coextruded rod of material having a higher moduls material on the outside and lower modulus material on the inside can be compression molded to produce a valve with improved seal surfaces by virtue of the lower modulus material on the inside and higher closure forces due to the high modulus material on the outside. The thickness of the two layers of material would depend upon the slit size and relative hardness of the inner and outer materials.

A flat or curvilinear apex may be used to make it easier to produce the slits in manufacturing. With the distal side of the valve coming to a point it is difficult to get a perfect cut at the apex, especially with a soft elastic material. The resulting slit can be incomplete or offset, causing the valve not to close properly or a small chunk of uncut material to remain at the apex that could break off and create an embolus.

Slits in the valves typically number from one to ten or more. To provide better separation of flaps in the open condition, it is preferred to have at least three slits running through the valve between the distal and proximal surfaces of the valve. Slits are typically two-dimensional or sheet-like cuts through the valve material, e.g., as is commonly understood. Slits can be planar boundaries between valve flaps. Slits can be three dimensional, e.g., described by curving surfaces. A single one-dimensional hole or path is not a slit.

In many embodiments, the valve slits on the distal surface run from the most distal aspect of the distal surface to a point on the distal surface adjacent to the pivot region. On the proximal side of the valve slits typically run from the center (typically most distal) of the second recess to the pivot region at the outer extent of the shoulder in the first recess. Of course, the slits include sheet-like cuts through the valve between the described surface features. Such complete slits can define functional flaps in the valve. Slits can run, e.g., from 1) a line defined by the most distal point of the distal surface and the most distal point of the second recess, to 2) a line defined by a point where the distal surface contacts the catheter bore wall with the valve in the closed position, and a point on the shoulder surface of the first recess. The slit between these two lines can be any functional shape. However, it is preferred the slit be planar or a curved surface.

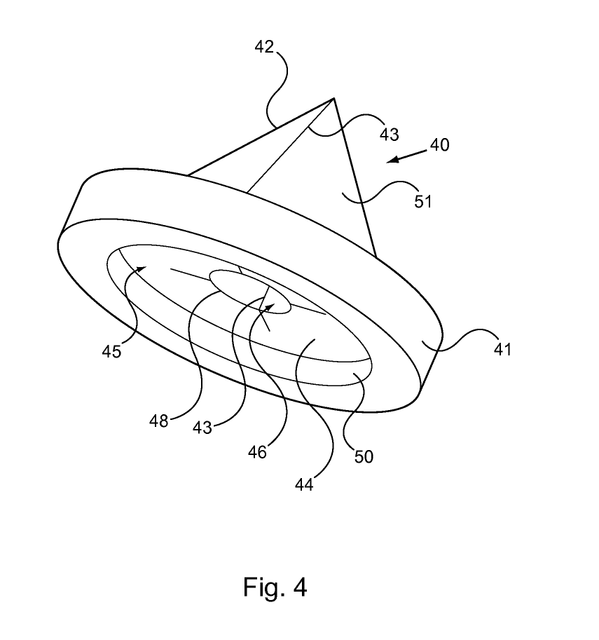

In some embodiments, it is preferred the slit run laterally on the proximal surface to at least the intersection of the first and second recesses, e.g., to provide a tight hermetic seal around a dilator or needle inserted through the second recess. Such a configuration can also provide a broad sealing surface to contact an inserted conduit (e.g., male luer). Alternately, the slits can run to a point between the intersection of the first and second recesses, e.g., to a point on the shoulder. This can allow freer movement of the flap bases as the valve is forced open. Optionally, the slits can terminate in the second recess without reaching the shoulder between the first and second recesses, e.g., for a tighter seal on the needle or a stronger return force to the normally closed position (e.g., as shown in FIG. 4). In preferred embodiments, the slits do not run laterally past a point on the surface of the first recess through which a tangential plane would intersect the axis of the catheter with an angle more than 45 degrees from the perpendicular plane at the point of axis intersection. In some embodiments, the slits run to at least the outer edge of the first recess.

Flaps defined by the slits are typically directed toward the distal end of the catheter. The outer surface of the flaps typically comprise valve distal surfaces. The inner surface of the flaps typically include surfaces defined by the slits and second recess. Because the slits, second recess, and shoulder surfaces typically run in different directions, the inner surface of the flaps are typically not simple curved surfaces, and most of the inner flap surface does not typically parallel the immediately adjacent outer flap surface, e.g., as shown in the present figures.

Recesses are depressions in a surface, as described herein. The edge of a recess is as one would ordinarily understand the edge to be, e.g., at a distinctly identifiable transition between the recess and the surface into which the recess is depressed. In many cases, the angle between the plane of the surface and a line described by a point on the outer wall of the recess to the closest point on the plane is about 90 degrees. For example, the angle at surface intersections (annular shoulder or step) between a first and second recess is often about 90 degrees, e.g., as shown in FIG. 4. However, the step angle between the first and second recesses can range from less than 45 degrees to more than 135 degrees, from 60 degrees to 120 degrees, from 75 degrees to 105 degrees, or about 90 degrees. These same ranges can apply to the angle between the outer proximal surface or flange of the valve and the first recess. Often, there is a point of inflection at or near the boundary edge between a first and second recess. Where the intersections between the surfaces are rounded, or otherwise not sharply angled, the angles can still be determined according to lines running from the surfaces approaching the intersection.

The first recess, excluding the second recess, typically has a shape complimentary to the end of a conduit intended to be received; particularly, complimentary to the conduit end and outer circumference near the conduit end. In many cases, the first recess is generally cylindrical in shape. The intersection of the first recess cylinder end, represented, e.g., by the shoulder feature, typically intersects at approximately a right angle with the sides of the recess. However, the first recess sides may be flared out somewhat (at an angle greater than 90 degrees), e.g., to swage, center, and seal a conduit tip introduced therein. In most embodiments, the primary seal between an introduced conduit and the valve is between the conduit end and the valve shoulder surface. Optionally, the side surface of the first recess can be configured to seal against the outer surface of the conduit, e.g., back from the conduit end. Optionally, in some embodiments, there is no “first recess” and the “second recess” originates from the bottom (proximal) end of the valve.

The second recess is typically radially symmetrical, e.g., hemispherical, cylindrical, or conical in shape. Optionally, the recess can be other shapes, such as cubical. The diameter of the second recess is typically about the same as the inner diameter of the conduit intended to open the valve by contacting the valve shoulder. It is preferred the distal end of the second recess comprise a tapered shape, e.g., to facilitate insertion of a needle or dilator, and to minimize turbulent fluid flows when in the valve is in an opened position.

Guide Needles

Guide needles are typically employed in the catheter assemblies for catheter insertion to provide a central rigid structure with a piercing tip functioning to provide confident control in piercing of skin and a vessel wall. Further, the guide needle typically provides a support structure or path to lead a dilator and/or catheter into the vessel. Guide needles can include a hub configured, e.g., for visual confirmation of vessel entry, interaction with external devices and/or positioning control relative to other device components. Alternately, catheters of the invention can perform the piercing function without a guide needle.

The catheter assemblies, before use and during insertion into a vessel, can have the needle running through a resilient valve in the hub of the catheter. Usually, the outer edge of the second recess has about the same diameter as the needle (and/or optional dilator), and the valve slits typically do not run outward substantially further than the needle/dilator diameter. When the needle/dilator is withdrawn from the assembly, the valve returns to the normally closed position, thus preventing fluids from flowing from the catheter hub.

Guide needles are usually rigid hollow structures with a pointed piercing distal end. In most embodiments of the invention, the guide needle is slidably mounted through the valve and within a dilator and/or catheter. In some cases, the distal end of the valve second recess is shaped to receive the beveled (e.g., slightly off center) needle tip without tearing during assembly of the catheter unit. (Optionally, pressure can be provided at the valve shoulder during manufacture to open the valve flaps during insertion of the needle through the valve.) Guide needles are typically cylindrical conduits with a circular cross section, or optionally can have cross sections of other shapes. The guide needles can be made from, e.g., stainless steel, a glass, ceramic, rigid plastic, and/or the like. Guide needles can range in length, e.g., from more than about 20 cm to about 0.5 cm, 10 cm to about 1 cm, from about 7 cm to about 2 cm, from about 5 cm to about 3 cm or about 4 cm. The guide needles can have an outer diameter (e.g., in the slidably mounted or piercing section) ranging, e.g., from more than about 2 cm to about 0.5 mm, from about 1 cm to about 0.6 mm, from about 5 mm to about 0.7 mm, from about 2 mm to about 0.8 mm, or about 1 mm. In many embodiments, the guide needle can essentially have the structure of a cannula or a hypodermic needle, e.g., ranging in size from 5 gauge to 34 gauge, from 8 gauge to 30 gauge, from 10 gauge to 28 gauge, from 12 gauge to 24 gauge, or about 22 gauge.

Guide needles can have a piercing end configured to pierce structures, such as skin, wall structures, membranes, vessel walls, and the like. The typical piercing end is a pointed beveled end, such as those used for hypodermic needles. In some embodiments, the beveled tip can include two or more sections with different bevel angles. In alternate embodiments, the guide needle can be hollow with a central slidably mounted wire having a conical piercing tip or the needle can be solid with a conical piercing tip. In many embodiments, it is preferred the guide needle have a central axial lumen so that entry into a vessel can be detected as vessel fluid appearing at the proximal end of the needle.

Guide needles commonly have a hub structure at the proximal end. Hubs typically have a greater inner diameter and/or outer diameter than the more proximal sections of the needle. In one embodiment, the needle hub is a clear chamber or “flash cup” flaring out from the proximal end of the needle, e.g., so that fluids can be viewed passing to or from the needle bore. In some embodiments, the chamber can include a gas vented membrane to prevent escape of liquid fluid from the proximal end of the needle. The needle hub can include fittings, such as a luer lock structure for connection to external devices, such as syringes. It is envisioned that needle hubs can include a resilient valve of the invention, as described herein.

The guide needle hub can optionally provide structures that interact with proximal hubs of the device dilator and/or catheter. For example, the needle hub can include tangs, grooves or cavities that interact with other hub structures to control or limit movement of the needle relative to other device structures. In some embodiments, the needle hub can have a structure configured to receive a mechanical force or pressure, e.g., intended to cause the needle to retract within a dilator and/or catheter.

Guide Dilators

Guide dilators are typically employed in the devices for catheter insertion to provide a dilating structure slidably mounted over a guide needle and having an outer diameter expanding away (tapered) from the distal tip. Such a structure can smoothly and painlessly enlarge a hole in a vessel wall initially made by the guide needle. In many embodiments, the guide dilator provides a support structure or path to lead a catheter into the vessel. Guide dilators can include a hub configured, e.g., for interaction with external devices and/or for positioning control relative to other device components.

Guide dilators are typically flexible or resilient hollow structures with a tapered distal end. In most embodiments of the invention that include dilators, the guide dilator is slidably mounted over a guide needle and also slidably mounted within a catheter. In the catheter assembly, the dilator is typically inserted through a resilient valve in the catheter hub. The valve can seal against fluid flows out of the catheter during insertion of the catheter into a vessel and when the dilator has been withdrawn from the catheter.

The guide dilators can be made from a flexible material, such as, e.g., silicone rubber, polypropylene, rubber, fluorocarbon plastics, polyurethane, and the like. In other embodiments, the dilator can be made from rigid materials. The guide dilator can be opaque or optionally translucent or transparent, e.g., to allow viewing of blood in the device lumen. Guide dilators can fit closely over guide needles of the device, e.g., touching the needle, functionally sealed over the needle, and/or within a small distance (e.g., spaced less than 20 um) from the needle. Guide dilators can range in length, e.g., from about 15 cm to about 0.7 cm, 10 cm to about 1 cm, from about 7 cm to about 2 cm, from about 5 cm to about 3 cm or about 4 cm. The guide dilators can have an inner diameter (e.g., in the section slidably mounted over the needle) ranging, e.g., from about 2 cm to about 0.5 mm, from about 1 cm to about 0.6 mm, from about 5 mm to about 0.7 mm, from about 2 mm to about 0.8 mm, or about 1 mm. In many embodiments, the dilator has a wall thickness configured to expand a vessel entry hole. The distally thin dilator wall can thicken proximally to a thickness ranging, e.g., from about 0.1 mm to about 1 cm, from about 0.5 mm to about 5 mm, from about 0.75 mm to about 2 mm, or about 1 mm.

A lubricant material can be applied to the inner surface of the dilator lumen and/or the needle outer surface to enhance sealing and/or reduce friction between the device components. The lubricant can include, e.g., silicone oil, silicone grease, mineral oil, vegetable oil, and/or the like.

Guide dilators can have a tapered distal end configured to dilate structures, such as skin, wall structures, membranes, vessel walls, and the like. In preferred embodiments, the tapered distal tip is relatively thin walled and closely contacts or seals over the outer surface of the needle distally. The wall thickness (and outer dilator wall diameter) progressively increases proximally from the tip. In many embodiments, the dilator outer diameter reaches a desired size (e.g., about the inner diameter of an associated catheter) and continues proximally for some distance with the same outer diameter. The distance from the tapered distal tip of the dilator to the final maximum distal outer diameter (dilator tapered section) typically ranges from about 30 cm to about 1 mm, from about 20 cm to about 2 mm, from about 10 cm to about 2 mm, from 7 mm to about 3 mm or about 4 mm.

Guide dilators often have a hub structure at the proximal end. The hub typically has a greater inner diameter and/or outer diameter than the more proximal sections of the dilator. In some embodiments, the chamber can include a valve or resilient membrane to seal the needle in use and/or to seal the inner bore of the dilator from the external environment should the needle be withdrawn from the device. The dilator hub can include fittings, such as a luer lock structure for connection to external devices, such as syringes.

The guide dilator hub can optionally provide structures that interact with proximal hubs of the device needle and/or catheter. For example, the needle hub can include tangs, grooves or cavities that interact with other hub structures to control or limit movement of the needle or dilator relative to other device structures. The dilator hub can nest in the catheter hub and/or provide a position in which a needle hub can nest. It is envisioned that dilator hubs can beneficially include a resilient valve of the invention, as described herein. In some embodiments, the dilator hub can have a space holding, e.g., a spring element under tension or expandable material, e.g., to provide a working mount and working force to actuate a needle retraction into the dilator.

IV-Catheters

Catheters of the inventive devices are, e.g., working devices and/or access ports intended for insertion into a vessel. The catheters are typically slidably mounted over the guide dilator and/or guide needle of the device and have an outer diameter expanding away (tapering) from the distal catheter tip. The catheter typically also has constant diameter conduit body proximal to the tapered tip. Such a structure can smoothly and painlessly further enlarge a hole in a vessel wall initially made by the guide needle and expanded by the dilator. Alternately, the catheter has a piercing end and provides its own entry into a vessel. In many embodiments, a rigid or flexible catheter can be guided through a vessel wall and/or some distance along the vessel lumen following the path of the guide dilator and/or needle. Catheters can include a hub configured, e.g., for interaction with external devices and/or for positioning control relative to other device components. The hub typically includes a resilient valve functioning to seal against fluid flows from the catheter during insertion and after needles and/or dilators have been withdrawn. The valve in the catheter hub can be configured, as described above, to allow access by contact with the end of an external device conduit.

Catheter components of the devices are typically flexible or resilient hollow structures with a tapered distal end. In many embodiments of the invention, the catheter is slidably mounted over a guide dilator or needle. The catheters can be made from a flexible material, such as, e.g., silicone rubber, polypropylene, rubber, fluorocarbon plastics, polyurethane, and the like. In other embodiments, the catheter can be made from rigid materials, such as stainless steel, a glass, ceramic, rigid plastic, etc. The catheter can be opaque or optionally translucent or transparent, e.g., to allow viewing of blood in the device lumen. Catheters can fit closely over guide dilators or needles of the device, e.g., touching the dilator, functionally sealed over the dilator, or within a small distance (e.g., spaced less than 20 um) from the dilator or needle outer surface. A lubricant can be present between the catheter and dilator or needle. Catheters can range in length, e.g., from more than about 15 cm to less than about 0.7 cm, 10 cm to about 1 cm, from about 7 cm to about 2 cm, from about 5 cm to about 3 cm or about 4 cm. The catheters can have an inner diameter (e.g., in the section slidably mounted over the dilator or needle) ranging, e.g., from more than about 3 cm to less than about 0.4 mm, from about 2 cm to about 0.5 mm, from about 1 cm to about 0.6 mm, from about 5 mm to about 0.7 mm, from about 2 mm to about 0.8 mm, or about 1 mm. Outer diameters and lengths of the catheter are typically greater for trocar embodiments than for IV embodiments. Catheter wall thickness is typically configured to suit the intended function of the catheter. The catheter wall typically ranges from about 0.1 mm to about 1 cm, from about 0.5 mm to about 5 mm, from about 0.75 mm to about 2 mm, or about 1 mm.

Catheters of the invention usually have a tapered distal end configured similarly to the dilator component for further dilation of structures, such as skin, wall structures, membranes, vessel walls, and the like. In preferred embodiments, the tapered distal catheter tip is relatively thin walled and closely contacts or seals over the outer surface of the dilator or needle distally. Optionally, the catheter has a piercing distal end, e.g., configured for venipuncture without the need of a separate needle. The wall thickness (and outer catheter diameter) can progressively increase proximally from the tip for some distance. In many embodiments, the catheter outer diameter reaches a desired size (e.g., for performance of the desired catheter function) and continues proximally for some distance with the same outer diameter. The distance from the tapered distal catheter tip to the final maximum distal outer diameter (catheter tapered section) typically ranges from about 30 cm to about 1 mm, from about 20 cm to about 2 mm, from about 10 cm to about 2 mm, from 7 mm to about 3 mm, or about 4 mm.

Catheters usually have a hub structure at the proximal end. The catheter hub typically has a greater inner diameter and/or outer diameter than the more proximal sections of the catheter. In some embodiments, a chamber of the catheter hub can include a resilient valve or resilient membrane to seal the dilator or needle in use and/or to seal the inner bore of the catheter from the external environment should the dilator be withdrawn from the device. The valve can include structures, such as a first and/or second recess that can allow access to the catheter bore by simple pressure from an external conduit tip (e.g., a male luer fitting). The catheter hub can include fittings (such as, e.g., a female luer lock structure) for connection to external devices, such as syringes, IV fluid conduits, surgical devices, electrodes, diagnostic devices, and/or the like.

The catheter hub can optionally provide structures that interact with proximal hubs of the device needle and/or dilator. For example, the catheter hub can include tangs, grooves or cavities that interact with other hub structures to control or limit movement of the needle or dilator.

Intravenous (IV) Lines

IV lines are conduits that provide a flow path to a catheter for infusion to a patient. In many instances, a catheter is emplaced to provide a continuous drip of fluids into a patient. The IV line can be attached on the proximal end to an IV fluid bag, typically held above the vessel for passive infusion into a patient. The continuous drip can provide the patent with required electrolytes, nutrients and drugs. The continuous drip also prevents occlusion, ensuring immediate access to the vessel.

Typically, an IV line will include a “Y” fitting with a septum allowing access to the IV flow, e.g., using a needle and syringe. Although this feature is optional in the present systems, it is not necessary to ensure access to the catheter. For example, the IV line can be connected to a catheter of the invention using a male luer fitting. The male luer tip is forced into the shoulder of the resilient valve and seated into a female luer fitting of the catheter. The luer tip force urges the valve flaps out against the inner catheter hub walls and provides an unobstructed fluid flow path from the IV line into the catheter and the vessel. When a sample of fluid is desired from the vessel, or when it is time to change the IV fluid bag, the IV line male luer is withdrawn from the catheter hub, removing the pressure of the luer tip from the valve shoulder. The valve closes as the luer tip is withdrawn. No fluid can escape from the catheter. The catheter and valve are configured so that insertion of a new external device luer tip can occur without entrapping any air in the fluid flow path. The new device opens the valve and fluids can again be injected or withdrawn from the vessel.

Vessels

The catheter assemblies of the invention are generally intended for use in placement of a catheter in a blood vessel. However, devices of the invention, e.g., provided in the appropriate range of sizes and materials, can facilitate insertion and/or placement of conduits through various barriers. For example, the “catheter” can be a trocar providing an access port for laparoscopic investigations or minimally invasive surgeries. The catheter can enter a vessel and progress within the vessel to a desired location some distance from the insertion point, e.g., for organ imaging, angioplasty or stent placement. In the most common embodiment, the “catheter” is essentially a semi-rigid large bore hypodermic conduit placed in a vein for fluid replacement and drug administration. In alternate embodiments, the “vessel” is not a part of a living organism.

In most cases, the vessel penetrated by the device is a conduit through which a fluid passes. For example, the vessel for catheter placement can be a vein, an artery, a lymph vessel, a portal vessel, or a gland duct. Optionally, the vessel can be a portion of a gastro-intestinal tract, respiratory tract, or a cerebral-spinal fluid compartment. The vessel can be a body compartment, such as, e.g., an ocular chamber, peritoneum, synovium, tympanum, and the like. Optionally, the devices of the invention can be used to gain access to channels or compartments not associated with animals, such as, e.g., plant vessels and chambers, or mechanical equipment chambers or conduits.

### Methods of Inserting of Inserting of Inserting of Inserting Catheters and Accessing Fluids

The present methods of inserting catheters and gaining access to the catheter bore generally include steps of inserting a catheter assembly, removing the needle and dilator, and accessing the catheter by pressing a conduit end to the shoulder of the catheter valve. For example, the methods can include inserting the distal piercing end of a guide needle through a patient's skin and through the wall of a blood vessel. The catheter-inserting device can be urged distally by the technician so that the distal tapered end of the dilator wedges into the vessel wall hole made by the needle and progresses to expand the hole to a larger diameter. The guide needle can withdrawn, e.g., at any time after the wedging of the dilator in the vessel. The tapered tip of the catheter can be urged distally onto the vessel wall hole and progress to expand the hole to receive the cross section of the main catheter body. The dilator can be withdrawn after the tip of the catheter has entered the vessel. With the needle and dilator withdrawn completely from the catheter hub, the catheter valve can resume the normally closed position, preventing any fluids from leaking out the proximal end of the catheter. Thereafter, one can gain access to the catheter bore and vessel by pressing a matching conduit onto the catheter valve shoulder to urge open the valve flaps, thereby providing a smooth, low resistance fluid flow path between the vessel and the conduit. Fluids can then be injected into the vessel from the conduit without also introducing air or residual fluids from any dead space in the catheter assembly. The conduit can be removed, releasing pressure on the valve shoulder and allowing the valve to assume the sealed normally closed position.

Providing the Catheter Insertion Device

The methods of inserting a catheter can be practiced using the catheter assemblies, e.g., as described herein. Briefly, insertion devices can be provided with a guide needle slidably mounted within a cylindrical guide dilator, which is slidably mounted within a cylindrical catheter. The three components can each comprise a tapered distal tip and/or a proximal hub. Any of the hubs can include a resilient valve; most preferably the catheter hub. The tapered tips can be configured to pierce and/or dilate a hole in the wall of a vessel. The hubs can be configured to accommodate technician handling of the device, control relative movement of the three components and/or functionally interact with external devices. The assembly can optionally exclude the needle and/or the dilator.

The catheter hub can include, e.g., a valve structure comprising a tapered slit distal surface, with the slits extending to the proximal side to the valve. The proximal side surface can have a first relatively large recess comprising a smaller second recess. A shoulder is defined by the boundary of the second recess on the first recess. The shoulder functions as an annular action arm that when pressed levers out valve flaps defined by the valve slits.

In a preferred embodiment, provision of a catheter insertion device includes assembly of a device by sliding a needle into a dilator so that the piercing end of the needle extends out from the distal end of the dilator. The dilator can be slid into the catheter through the resilient self-closing valve so that the dilator outer surface is hermetically sealed in the catheter hub and the tapered tip of the dilator extends out from the distal end of the catheter. In use, the distal ends of the needle, dilator and catheter are inserted into a blood vessel, the needle is retracted, and the dilator is withdrawn while the inner aspects of the catheter are sealed by the valve from the external environment. External devices can be attached to any of the proximally remaining needle, dilator or catheter. Any of the three elements can have a resilient valve, as described above, and avail access to the central bore of the assembly. In preferred embodiments, at least the catheter hub includes a self-closing access valve.

Inserting the Device

Methods of placing and accessing a catheter include, e.g., steps of inserting the three components (needle/dilator/catheter) into a vessel. The guide needle functions to make the initial pierced hole in the skin or vessel wall. The dilator can follow the needle to expand the size of the hole to allow entry of the catheter and/or can be structured to function as a guide to direct the catheter some desired distance within the vessel. The catheter is typically inserted last and can further expand the hole and/or can be designed to remain in place within the vessel after the needle and/or dilator are removed from the vessel. In alternate methods, the catheter (e.g., with resilient valve in hub) is inserted without accompanying needle and/or dilator.

The piercing end of the needle can be inserted into the wall of a vessel, e.g., in a manner similar to insertion of a hypodermic needle or old art catheter. Typically the piercing end of the guide needle is inserted through a patient's skin at a point overlying a blood vessel to be catheterized. The dilator and catheter can follow before piercing the vessel, but the needle typically pierces the vessel before the catheter enters the skin. The guide needle acts as an insertion guide for the dilator and in many cases the needle has pierced both the skin and vessel before the dilator has entered the skin. Because the guide needle is rigid, it provides the technician with a topological certainty and structural strength required to confidently manipulate the device and complete the required mechanical task of entering the intended vessel.

The guide dilator is supported and directed by the guide needle for insertion into the vessel and for dilation of the entry hole. Once the dilator has entered the vessel, the needle can be retracted so that the piercing end is covered by, e.g., softer and more resilient material of the dilator to avoid piercing of an opposite vessel wall by the needle. In some embodiments, the needle is initially only retracted to within the dilator, but not retracted to a point outside the vessel. With this arrangement, the needle can continue to provide a rigid tool for the technician to manipulate progression of the dilator and provide solid backing to the dilator as it dilates the vessel hole to a larger diameter. In some embodiments, the needle can be held at a point within the vessel as the guide dilator slides distally to progress further into the vessel. In this way, a solid structural presence is maintained at the entry hole while the flexible dilator body progresses along the vessel, e.g., to provide a path of later insertion of the catheter. Alternately, the needle can be withdrawn entirely out of the vessel and/or entirely from the device before the dilator has completed progression and/or before the catheter has entered the vessel.

The catheter can be inserted into the vessel while the guide needle and/or guide dilator remain inserted through the vessel at the initial insertion point. The catheter can be inserted into the vessel while the distal tip of the guide needle and/or distal tip of the guide dilator are just inside the vessel and/or after a distal tip has been inserted some distance along the interior of the vessel. In a preferred embodiment, the needle is inserted some distance within the vessel and the guide dilator is just inside the vessel when the catheter is inserted through the vessel wall. In a preferred embodiment, the catheter is inserted through the vessel wall with both the guide dilator and the guide needle inserted some distance (e.g., 1 cm, 2, cm, 5 cm 10 cm or more) along the vessel. In a more preferred embodiment, the catheter is inserted through the vessel wall while both the guide needle and guide dilator are just inside (e.g., not having progressed more than 2, 5 or 10 dilator outer diameters) the vessel. In a most preferred embodiment, the catheter is inserted through the vessel wall while the guide dilator has been inserted some distance along the vessel and the guide needle is just inside the vessel. In this way, the catheter has solid support to enter the vessel but resilient support to progress along a curving path of a fragile vessel.

Embodiments where the needle is not inserted as far as the dilator can be accomplished by slidable retraction of the needle to a point within the dilator, or by complete withdrawal of the needle while the dilator remains in the vessel. With the needle retracted, the flexible dilator tip can facilitate progression along the vessel while minimizing the likelihood of trauma to the vessel interior.

In many embodiments, the catheter assembly does not include a dilator, but simple a needle slidably mounted within the catheter. In such a case, the piercing end of the needle can be inserted into the wall of a vessel, e.g., with the guide needle acting as an insertion guide for the catheter. The catheter can then be inserted into the vessel while the guide needle a remains inserted through the vessel at the initial insertion point. The catheter can be inserted into the vessel while the distal tip of the guide needle is just inside the vessel and/or after a distal needle tip has been inserted some distance along the interior of the vessel. In a preferred embodiment, the needle is inserted some distance within the vessel when the catheter is inserted through the vessel wall.

Once the needle and dilator are withdrawn, the valve provides a sanitary, low volume, accessible seal to the proximal catheter. The free flow of the catheter can be tested by attaching a syringe to the catheter hub, pressing the valve shoulder with the syringe male luer tip, and drawing back the syringe plunger. If the catheter is properly installed, fluid will flow freely from the vessel, through the catheter body, across the open resilient valve and into the syringe. Fluid samples can be obtained at this point. Next, the syringe can be withdrawn from the catheter and the catheter hub will self seal. An IV line end can be inserted into the catheter hub to open the valve and allow IV fluids to infuse into the patient's blood vessel.

### EXAMPLES

The following examples are offered to illustrate, but not to limit the claimed invention.

### Example 1

### A Catheter Insertion Assembly

An exemplary catheter assembly was manufactured including a guide needle for perforation of skin and vessel; a guide dilator to expand the needle perforation, protect the vessel from further perforations and to guide a catheter into the vessel; and, a catheter to provide access to the vessel by clinical technicians. The assembly included a resilient valve, e.g., in the catheter hub and traversed by the needle and dilator.

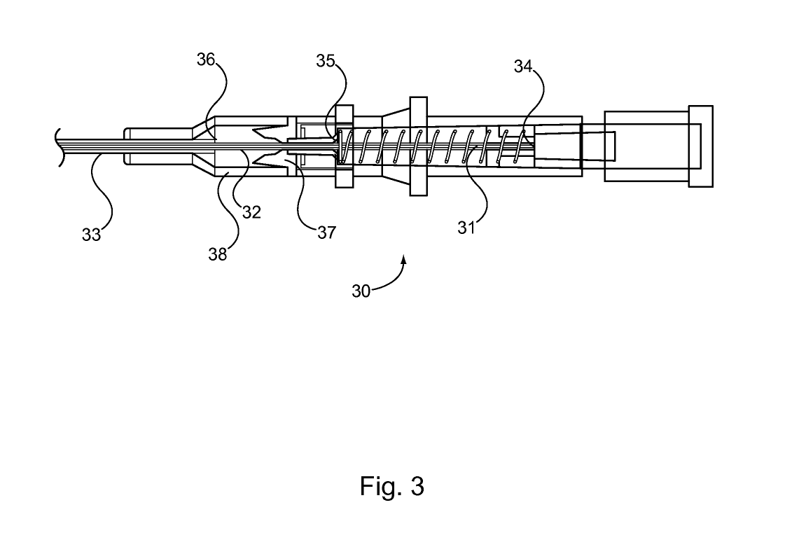

FIG. 3 shows a catheter insertion assembly 30 composed of a guide needle 31, typically formed from stainless steel; a guide dilator 32, typically a tough, flexible plastic such as polyurethane or polytetrafluoroethylene; and, an intra-vascular catheter 33, also produced from a tough, flexible material, and of a geometry needed for a given medical procedure. These three components of the invention were fitted together concentrically such that the proximal end 34 of the guide needle protruded from the proximal end 35 of the guide dilator, and the guide dilator protruded from the proximal end 36 of the intra-vascular catheter. Self-closing resilient valve 37 was mounted in catheter hub 38 with the dilator and needle running through past the second recess and valve slits.

Catheter assemblies manufactured with the valve held open, away from the dilator/needle can maintain proper sealing surfaces in storage, until they are needed during use. Note that in FIG. 3, the valve is urged open by the end of a cylindrical collar around the dilator. It can be desirable to retain the valve in an open position before use, while the catheter assembly is in storage. For example, the needle and/or dilator hub could be shaped like a male luer so that when fully inserted in the catheter, the valve is pushed open by the needle/dilator hub, e.g., during assembly of the device. This configuration can prevent the valve from losing the integrity of its seal surfaces by avoiding permanent setting and deformation to an undesirable shape from viscoelastic compression of the valve flaps during extended contact, e.g., with the dilator and/or needle during storage. As the needle/dilator is withdrawn, the hub would lose contact with the valve shoulder, allowing the undistorted valve flaps (or the intersecting point of the first and second recesses) to close against the needle/dilator, re-establishing the seal until the needle/dilator is fully withdrawn. With the dilator/needle fully withdrawn, the valve can close with sealing surfaces undistorted by contact during storage.

### Example 2

### Valve Configurations

FIG. 4 shows an oblique view of the one way valve 40. The valve is composed of an outer flange 41, where it is held in position in the proximal hub of a catheter. The valve includes generally conical shaped distal surface 42, slits 43, and proximal surface 44. The proximal surface includes a first recess 45 and second recess 46, defining shoulder 48 where the edge of the first recess meets the second recess.

Note that slits 43 run from the apex of the tapered distal surface, through the body of the valve to the proximal side, ending at a point between the shoulder 48 and the outer wall 50 of the first recess 45. In this example, the valve flaps 51 comprise most of the valve body except the flange 41.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A self-closing valve comprising:
a tapered distal surface;
a proximal surface with a first recess;
a second recess in the first recess distal to the first recess; and,
one or more slits running from the tapered distal surface to the second recess.

2. The valve of claim 1, wherein the self-closing valve comprises a resilient material.

3. The valve of claim 1, wherein the tapered surface is conical and the slits comprise three or more radially arranged slits.

4. The valve of claim 1, wherein the proximal surface further comprises a flange around the first recess.

5. The valve of claim 1, wherein the first and second recesses comprise circular outer edges, and wherein the outer edge of the second recess defines a shoulder between the first recess and second recess.

6. The valve of claim 5, wherein the one or more slits run from a top center of the distal surface to at least the edge of the second recess where the second recess contacts the first recess.

7. The valve of claim 6, wherein the one or more slits run to a point between the shoulder and the outer edge of the first recess.

8. The valve of claim 1, wherein the valve is adapted so that a force exerted at an intersection of the first and second recesses forces open the one or more slits, thereby functioning to open a fluid flow path through the second recess.

9. The valve of claim 1, wherein a tapered distal surface comprises a flat or curvilinear apex.

10. A valve as shown in FIG. 4.

11. A catheter insertion assembly comprising:
a catheter comprising a hub at a catheter proximal end; and,
a resilient self-closing valve in the hub, the valve comprising a distal tapered surface and a proximal surface comprising a first recess, wherein the proximal surface comprises a second recess located on the surface of the first recess.

12. The assembly of claim 11, further comprising a flexible dilator or rigid needle slidably mounted within the catheter through the valve.

13. The assembly of claim 12, wherein the flexible dilator or rigid needle includes a collar adapted to press against a shoulder between the first recess and second recess, thus holding open the valve while the dilator or needle is mounted within the catheter.

14. The assembly of claim 12, wherein the resilient valve is adapted to close and seal to fluid flow when the dilator or needle is withdrawn from the valve.

15. The assembly of claim 11, wherein the second recess is located on the distal center of the first recess.

16. The assembly of claim 11, wherein one or more slits run from a top center of the valve distal surface to at least an outer edge of the second recess at a point where the second recess contacts the first recess.

17. The assembly of claim 11, wherein one or more slits comprises at least 3 slits in a radial pattern.

18. The assembly of claim 11, wherein the hub comprises a female luer fitting.

19. A method of accessing a vessel interior or bore of a first conduit, the method comprising:
providing a first conduit hub or vessel hub comprising a self closing resilient valve, the valve comprising:
a tapered distal surface;
a proximal surface comprising a first recess and a second recess, wherein the second recess is located in the first recess, the intersection of the first recess and second recess defining a shoulder; and,
one or more slits running from the distal surface to the second recess; and,
opening the valve by pushing an end of a second conduit distally onto the shoulder, thereby providing a fluid flow path between the bore or vessel interior at the distal surface and the second conduit at the proximal surface.

20. The method of claim 19, further providing the second recess with a circular outer edge centered in the first recess.

21. The method of claim 19, further providing the first recess with an outer edge on the proximal surface, and providing that the one or more slits further runs to a point between the shoulder and the outer edge of the first recess.

22. The method of claim 19, wherein the second conduit is provided with working surfaces of a male luer fitting.

23. The method of claim 19, wherein the vessel or first conduit is selected from the group consisting of: a catheter, a dilator, a chamber, a pipe, a hose, a storage tank, a carboy, a fuel tank, and a hydraulic line.
